(19) United States
(12) Patent Application Publication
(10) Pub. No.: US 2005/0131316 A1
(43) Pub. Date: Jun. 16, 2005

(54) Guidewire with flexible tip

(75) Inventors: Jacob A. Flagle, Indianapolis, IN (US); Thomas A. Osborne, Bloomington, IN (US)

Correspondence Address: J. Matthew Buchanan;Brinks Hofer Gilson & Lione, P.O. Box 10395, Chicago, IL, 60610, US

(73) Assignee: Cook Incorporated

(21) Appl. No.: 10/736,047

(22) Filed: Dec. 15, 2003

(51) Int. Cl. A61B 5/00; A61M 25/00

(52) U.S. Cl. 600/585

(57) ABSTRACT

A guidewire with a flexible tip is provided. The flexible tip comprises a portion of a coating that axially extends beyond a distal end of an elongate member of the guidewire. The length of the flexible tip can be defined in relation to a diameter of the elongate member of the guidewire. Also, the length of the flexible tip can be defined in relation to a length of a distal tip portion of the elongate member of the guidewire. The flexible tip may include agents that facilitate visualization, such as an opacifying agent in the coating and/or a radiopaque marker disposed in the flexible tip.

### FIELD OF THE INVENTION

The present invention relates to medical devices. More specifically, the present invention relates to guidewires for placement within a body vessel.

### BACKGROUND OF THE INVENTION

Minimally invasive medical procedures allow medical practitioners to conduct procedures inside a patient's body with minimal surgical trauma. Such procedures often involve the navigation of an endoluminal device, such as a catheter or other cannula, through a body vessel to a distant point of treatment. For example, a physician can reinforce a cardiac artery by implanting a stent in the artery. The physician can place the stent at the point of treatment by navigating a catheter with a mounted stent through the vasculature. Once the point of treatment is reached, the physician deploys the stent and subsequently retracts the catheter from the point of treatment and eventually from the patient's body. The entire procedure, therefore, is conducted through a small opening providing access to the vasculature as opposed to direct surgical access to the cardiac artery.

In minimally invasive procedures, a guidewire can be used to aid navigation of endoluminal devices through body vessels. Guidewires are typically elongate metal members with a distal tip having a protective means, such as a ball or soft coating, that minimizes trauma to the vessel lining during navigation of bends and other potential obstacles. Guidewires can be initially navigated through a vessel to provide an established route of access for subsequent endoluminal devices, such as catheters, that can be advanced over the placed guidewire. Also, a guidewire/catheter combination can be advanced through the vasculature as a single unit. Furthermore, upon retraction of a catheter, a guidewire can be left in place to provide an established track for subsequent access to a particular point of treatment using the same or another catheter.

### SUMMARY OF THE INVENTION

The present invention provides a guidewire with a flexible tip that axially extends beyond the distal end of the elongate body of the guidewire. A ratio of the length of the flexible tip to the diameter of the main body of the guidewire can be greater than 3:1. Also, the length of the flexible tip can be equal to or greater than the length of a distal tip portion of the elongate member of the guidewire.

In one embodiment, a guidewire according to the invention comprises an elongate member having a proximal end, a main body, a distal end, and a taper portion. The main body has a first diameter and the distal end has a second diameter. The taper portion is disposed between the main body and the distal end and defines a taper between the first and second diameters. A coating is disposed on the distal end and comprises a flexible tip. The flexible tip comprises a length axially extending beyond the distal end of the elongate member. The ratio of the length of the flexible tip to the first diameter is greater than 3:1.

In another embodiment, a guidewire according to the invention comprises an elongate member having a proximal end, a main body, a distal tip, and a taper portion. The main body has a first diameter and the distal tip has a second diameter. The taper portion is disposed between the main body and the distal tip and defines a taper between the first and second diameters. A coating is disposed on the distal tip and comprises a flexible tip. The flexible tip has a length that axially extends beyond the distal tip. The length of the flexible tip is equal to or greater than the length of the distal tip portion of the elongate member.

In another embodiment, a guidewire according to the present invention comprises an elongate member having a proximal end, a main body, a distal tip, and a taper portion. The main body has a first diameter and the distal tip has a second diameter. The taper portion is disposed between the main body and distal tip and defines a taper between the first and second diameters. A coating is disposed on the distal tip and at least a portion of the taper portion. The coating comprises a flexible tip having a length that axially extends beyond the distal tip of the elongate member. The ratio of the length of the flexible tip to the first diameter is greater than 3:1. A radiopaque marker is disposed in the flexible tip and a lubricious coating is disposed on at least a portion of the main body.

Additional understanding of the invention can be obtained from the description of exemplary embodiments appearing below and the appended drawings.

### BRIEF DESCRIPTION OF THE DRAWINGS

FIG. 1 is a side view of a guidewire according to one embodiment of the invention, with portions in cross section.

FIG. 2 is a magnified view of the distal end of the guidewire illustrated in FIG. 1.

FIG. 3 is a magnified view of a distal end of a guidewire according to another embodiment of the invention.

FIG. 4 is a side view of a guidewire according to another embodiment of the invention, with portions in cross section.

FIG. 5 is a magnified view of the distal end of the guidewire illustrated in FIG. 4.

FIG. 6 is a magnified view of the distal end of a guidewire according to another embodiment of the invention.

FIG. 7 is a side view of a guidewire according to another embodiment of the invention, with portions in cross section.

FIG. 8 is a side view of a guidewire according to another embodiment of the invention, with portions in cross section.

FIG. 9 is a side view of a guidewire according to another embodiment of the invention, with portions in cross section.

FIG. 10 is a sectional view of a guidewire according to another embodiment of the invention.

FIG. 11 is a magnified view of the distal end of the guidewire illustrated in FIG. 10.

### DETAILED DESCRIPTION OF EXEMPLARY EMBODIMENTS OF THE INVENTION

The following provides a detailed description of embodiments of the invention. The embodiments described and illustrated herein are exemplary in nature, and are not intended to limit the scope of the invention in any manner. Rather, the embodiments serve simply as examples to aid in enabling one of ordinary skill in the art to make and use the invention.

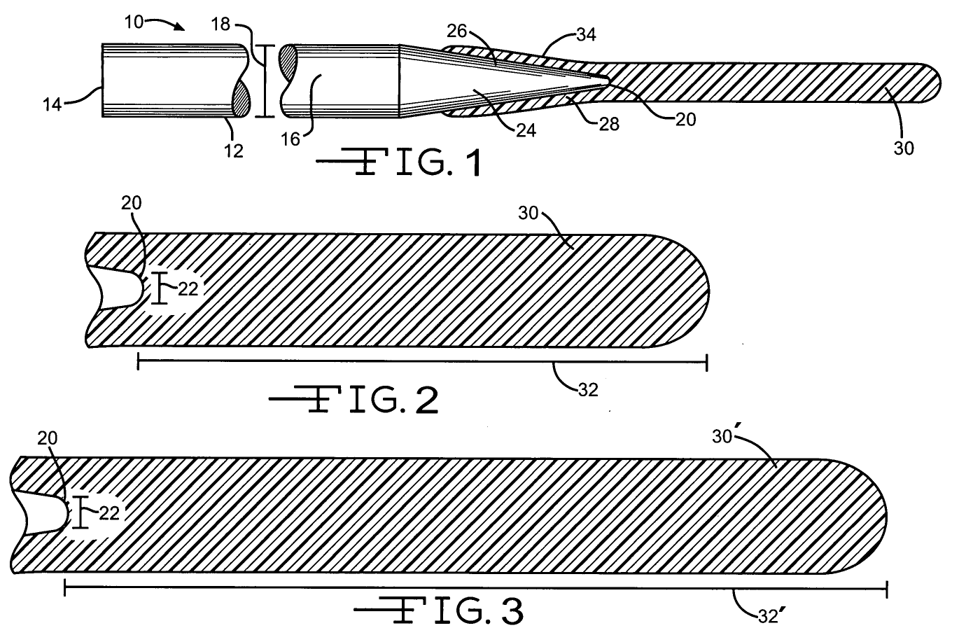

The present invention provides a guidewire with a flexible tip that axially extends beyond the distal end of the elongate body of the guidewire. FIGS. 1 and 2 illustrate a guidewire 10 according to one embodiment of the invention. In this embodiment, the guidewire 10 comprises an elongate member 12 that has a proximal end 14, a main body 16 with a first diameter 18, and a distal end 20 with a second diameter 22. A taper portion 24 is disposed between the main body 16 and distal end 20, and defines a taper 26 between the first 18 and second 22 diameters. A coating 28 is disposed on the distal end 20 of the elongate member 12. The coating 28 comprises a flexible tip 30 that has a length 32 extending axially beyond the distal end 20 of the elongate member 12. The coating 28 can be adhered to the elongate member 12 by way of its physical characteristics, an additional adhesive, over-molding techniques, or other suitable manners known in the art.

The elongate member 12 can comprise any member suitable for use as a guidewire. The guidewire art contains numerous examples of suitable elongate members, such as unitary wire members, wound wire members, and members comprising one or more wires wound around a mandril. Further, the elongate member 12 can comprise a unitary construction or two or more components arranged together to form a suitable elongate member.

The elongate member 12 can be fabricated from any suitable material, and need only be biocompatible or be able to be made biocompatible by techniques such as coating, chemical treatment, and the like. Examples of suitable materials include polymeric materials, such as polyethylenes and other plastics, and metals, such as stainless steel, nitinol, and other shape memory metals.

The elongate member 12 includes proximal 14 and distal 20 ends. During a procedure using the guidewire 10, the proximal end 14 of the elongate member 12 typically remains outside the patient and is used by the practioner to effect navigation of the guidewire 10 through a body vessel. Accordingly, the proximal end 14 may include adaptations and/or structures that aid in handling the guidewire 10, such as a handle or other gripping structure. The distal end 20 provides the leading end for navigation through a body vessel.

The elongate member 12 also includes a main body 16 between the proximal 14 and distal 20 ends. The main body 16 typically comprises the majority of the overall length of the elongate member 12. The main body 16 has a diameter 18 that may vary or remain constant along the length of the elongate member 12. The diameter 18 represents the finished diameter of the main body 16 and, therefore, includes any coatings, layers, or other materials placed on the exterior surface of the main body 16 to create the finished guidewire 16.

The diameter 22 of the distal end 20 can be smaller than the diameter 18 of the main body 16. The embodiment illustrated in FIGS. 1 and 2 includes a guidewire 10 having a distal end 20 with a diameter 22 that is smaller than a diameter 18 of the main body 16. With this arrangement, the elongate member 12 can include a taper portion 24 disposed between the main body 16 and the distal end 20. The taper portion 24 defines a taper 26 between the diameter 18 of the main body 16 and the diameter 22 of the distal end 20. The taper portion 26 can be integrally formed with the main body 16 and/or distal end 20, or can be a separately attached member. For example, a frusto-conical member can be attached to an end of a main body portion, thereby providing the distal end and taper portion of an elongate member for use in a guidewire according to the invention. The taper portion 24 provides a portion that accomplishes a reduction in diameter, and may enhance flexibility. The distal end 20 can comprise a rounded tip, as illustrated, or any other suitable configuration, including a pointed tip.

A coating 28 is disposed on the distal end 20 of the elongate member 12. The coating 28 can comprise any suitable coating, and need only be biocompatible or be able to be made biocompatible. Furthermore, the coating must be able to provide the flexible tip as described herein. Suitable materials for the coating 28 include polymeric materials, such as polyurethane and other polymers capable of being flexible at human body temperatures. If a polymeric coating is used, the coating 28 can comprise a single polymer or a blend of one or more polymers. The specific polymer(s) chosen will depend on several factors, including the desired flexibility of the coating 28 and flexible tip 30 (described more fully below) and the material used for the distal end 20 of the elongate member 12.

The coating 28 comprises a flexible tip 30. The flexible tip 30 is a portion of the coating 28 that axially extends beyond the distal end 20 of the elongate member 12. Thus, the flexible tip 30 does not include any portion of the elongate member 12. As best illustrated in FIG. 2, the flexible tip 30 has a length 32 that represents the distance by which the tip 30 axially extends beyond the distal end 20 of the elongate member 12. The length 32 can be defined in comparison to dimensions of the main body 16. For example, the length 32 can be defined as a particular ratio to the diameter 18 of the main body 16. When defined in this manner, the ratio of the length 32 to the diameter 18 can be greater than 3:1. Also, the ratio of the length 32 to the diameter 18 can be between 10:1 and 500:1, between 10:1 and 300:1, and between 12:1 and 250:1.

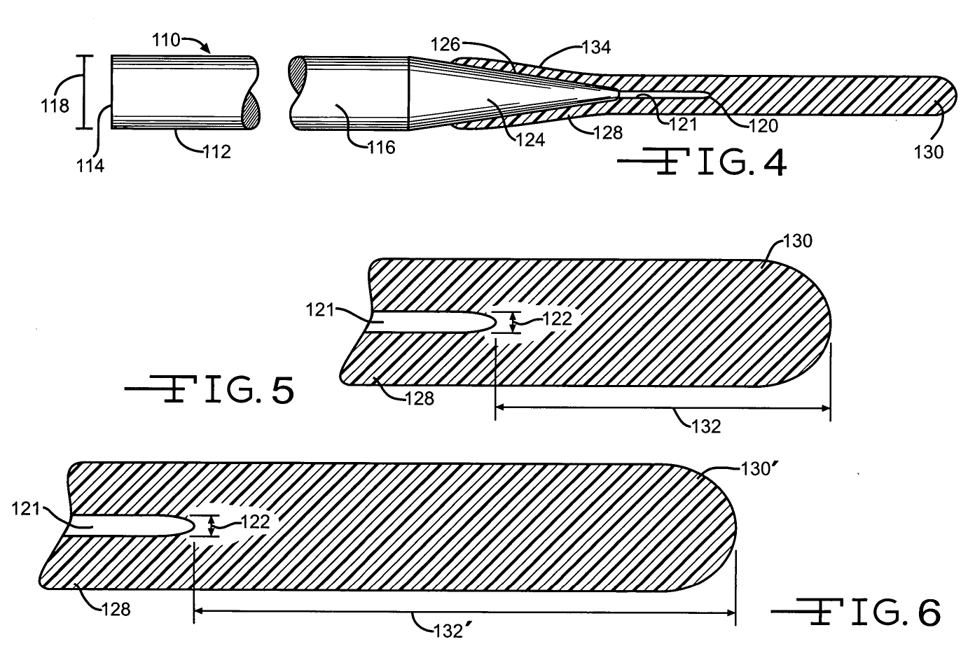

The length 32 of the flexible tip 30 can also be defined as a length measurement. The length 32 should be greater than 3 mm for most guidewires, and can be between 3 and 100 mm, between 4 and 80 mm, between 5 and 20 mm, and between 8 and 12 mm. A 10 mm length is a particularly well suited length for a flexible tip on a guidewire for use in some vascular applications. FIG. 2 illustrates a length 32 of the flexible tip 30 and FIG. 3 illustrates an alternate length 32′ of a flexible tip 30′.

The specific length chosen for the flexible tip 30 will depend on various factors, and can be optimized based on these factors. For example, the flexible tip 30 provides a flexible end to the guidewire 10, and may provide flow direction to the guidewire 10. That is, the flexible tip 30 can lead the guidewire 10 by following the flow of blood or other fluid through the body vessel in which the guidewire 10 is placed, which may aid in navigation into smaller vessels, such as distal arteries. A longer flexible tip 30 may better respond to curves and other turns and/or bifurcations in the vessel than a shorter flexible tip. Also, the length of the flexible tip 30 must not be so excessive as to negatively affect the pushability and/or steerability of the guidewire 10. Furthermore, the length of the flexible tip 30 can be optimized based on the intended use of the guidewire, such as the typical vessels into which the guidewire 10 will be placed.

For example, if a guidewire will be used to traverse a narrow stenosis in the peripheral vasculature, or if a guidewire will be used with large, bulky catheters or other devices, a relatively shorter tip may be desirable. On the other hand, if a guidewire will be used in distal, tortuous vasculature, a relatively longer flexible tip may be desirable. Longer flexible tips may be up to 100 mm or longer and may be associated with guidewires of relatively small diameter, such as guidewires having a diameter of 0.018″. Shorter flexible tips may be as short as 3 mm and may be associated with guidewires of relatively larger diameter, such as guidewires having a diameter of 0.038″.

In the embodiment illustrated in FIG. 1, the coating 28 is further disposed on part of the taper portion 24 of the elongate member 12. This may provide additional anchoring of the coating 28 to the elongate member 12. Also, the coating 28 in this embodiment includes a taper 34 that roughly approximates the taper 26 of the elongate member 12 taper portion 24.

FIGS. 4 and 5 illustrate a guidewire 110 according to another embodiment of the invention. The guidewire 110 of this embodiment is similar to the guidewire 10 illustrated in FIGS. 1 and 2, except as detailed below. Thus, the guidewire 110 includes an elongate member 112 having proximal 114 and distal 120 ends. A main body 116 extends between the proximal 114 and distal 120 ends. The main body 116 has a first diameter 118 and the distal end 120 has a second diameter 122. A taper portion 124 is disposed between the main body 116 and distal end 120 and defines a taper 126 between the first 118 and second 122 diameters. A coating 128 is disposed on the distal end 120 and comprises a flexible tip 130 that has a length 132 that axially extends beyond the distal end 120 of the elongate member 112.

In this embodiment, the elongate member 112 further includes a distal tip portion 121 disposed adjacent the taper portion 124. The distal tip portion 121 is an elongate tip of substantially uniform diameter 122 and comprises the distal end 120 of the elongate member 112. The distal tip portion 121 may provide additional rigidity to the distal end of the guidewire 110 and may enhance securement of the coating 128 to the elongate member 112.

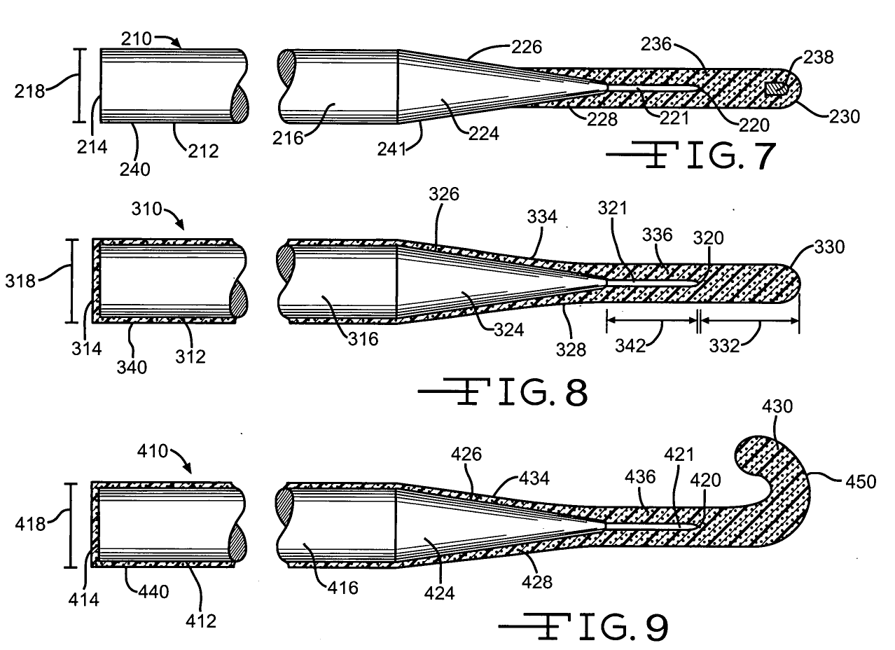

In this embodiment, the length 132 of the flexible tip 130 comprises the distance by which the coating 128 axially extends beyond the distal end 120 of the elongate member 112, which is the end of the distal tip portion 121. The length 132 of the flexible tip 130 can be defined by the same comparative ratios to the finished diameter 118 of the main body 116 and/or length values as in the embodiment illustrated in FIG. 1, described above. FIG. 6 illustrates an alternate flexible tip 130′ having alternate length 132′. The distal tip 121 has the same diameter 122 as in the embodiment illustrated in FIG. 5.

FIG. 7 illustrates a guidewire 210 according to another embodiment of the invention. The guidewire 210 of this embodiment is similar to the guidewire 110 illustrated in FIG. 4, except as described below. Thus, the guidewire 210 includes an elongate member 212 having proximal 214 and distal 220 ends. A main body 216 extends between the proximal 214 and distal 220 ends and has a diameter 218. A distal tip portion 221 is disposed adjacent a taper portion 224. The distal tip portion 221 comprises an elongate tip of a second, substantially uniform diameter and comprises the distal end 220 of the elongate member 212. The taper portion 224 is disposed between the main body 216 and the distal end 220 and defines a taper 226 between the first diameter 218 and the diameter of the distal tip portion 221. A coating 228 is disposed on the distal end 220 and comprises a flexible tip 230 that has a length that axially extends beyond the distal end 220 of the elongate member 212.

In this embodiment, the coating 228 further comprises radiopaque material 236. The radiopaque material 236 provides radiopacity to the coating 228, allowing enhanced visualization under fluoroscopy. The radiopaque material 236 can comprise any suitable opacifying agent loaded into the coating 228, such as bismuth, tantalum, or other suitable agents known in the art. The concentration of the agent in the coating 228 may require adjustment based on the size of the elongate member 212 and/or the coating 228. For example, guidewires with distal ends of relatively small diameter may require an 80% load of radiopaque material 236 to be adequately visible under fluoroscopy. Larger guidewires, however, may require only a 30% load.

A radiopaque marker 238 can be disposed in the flexible tip 230. The radiopaque marker 238 is a solid member disposed in the flexible tip 230 and spaced from the distal end 220 of the elongate member 212. Thus, as illustrated in FIG. 7, a portion of the coating 228 can be disposed between the radiopaque marker 238 and the distal end 220 of the elongate member 212. The radiopaque marker 238 can comprise any suitable radiopaque material, such as gold, platinum, or other suitable materials known in the art. The radiopaque marker 238 may provide additional visibility to the flexible tip 230 under fluoroscopy, and can be used in conjunction with or in the absence of radiopaque material 236. Also, as a solid member, the radiopaque marker 238 provides additional localized weight to the flexible tip 230, which may enhance the flow direction properties of the flexible tip 230, as described above. If only the additional localized weight provided by the marker 238 is desired, any suitable solid member can be used. For example, if only additional localized weight is desired in the flexible tip 230, a solid member that is not radiopaque can be used. In such embodiments, essentially any material that can be placed in the flexible tip 230 can be used, and suitable materials include metals and polymeric materials. Also, the radiopaque marker 238, or solid member that is not radiopaque, can be disposed around the flexible tip 230, or in any other suitable arrangement in or on the flexible tip 230.

In this embodiment, a lubricious coating 240 is disposed on at least a portion of the main body 216 of the elongate member 212. The lubricious coating 240 reduces friction during navigation of the guidewire 210 through a body vessel. A variety of lubricious coatings are known in the guidewire art, and any suitable coating can be used in accordance with the present invention. An example of a suitable coating is ePTFE. The lubricious coating 240 can be disposed on any portion of the main body 212, and may be disposed over the entire surface of the elongate member 212. Also, the lubricious coating 240 may be disposed on a portion of the taper portion 224 of the elongate member 212. For example, the lubricious coating 240 may be disposed on the part 241 of the taper portion 224 that is not covered by the polymer coating 228.

Also, in this embodiment, the coating 228 comprising the flexible tip 230 has a substantially uniform diameter along its length. Thus, in contrast to the embodiments described above, the exterior surface of the coating 228 does not include a taper that roughly approximates the taper 226 of the taper portion 224 of the elongate member 212. Rather, the exterior surface of the polymer coating 228 is substantially linear.

FIG. 8 illustrates a guidewire 310 according to another embodiment of the invention. The guidewire 310 of this embodiment is similar to the guidewire 110 illustrated in FIG. 4, except as described below. Thus, the guidewire 310 includes an elongate member 312 having proximal 314 and distal 320 ends. A main body 316 extends between the proximal 314 and distal 320 ends and has a diameter 318. A distal tip portion 321 is disposed adjacent a taper portion 324. The distal tip portion 321 comprises an elongate tip of a second, substantially uniform diameter and comprises the distal end 320 of the elongate member 312. The taper portion 324 is disposed between the main body 316 and the distal end 320 and defines a taper 326 between the first diameter 318 and the diameter of the distal tip portion 321. A coating 328 is disposed on the distal end 320 and comprises a flexible tip 330 that has a length that axially extends beyond the distal end 320 of the elongate member 312.

In this embodiment, the coating 328 is disposed on the distal end 320, the taper portion 324, and at least a portion of the main body 316. Also, a lubricious coating 340 is disposed on at least a portion of the coating 328. As illustrated in FIG. 8, the lubricious coating 340 can be disposed on the surface of the entire coating 328.

The length 332 of the flexible tip 330 can be defined by the comparative ratios and/or length values as in all previous embodiments described above. Also, in this embodiment, the length 332 of the flexible tip 330 can be defined as being equal to or greater than a length 342 of the distal tip portion 321 of the elongate member 312.

FIG. 9 illustrates a guidewire 410 according to another embodiment of the invention. The guidewire 410 of this embodiment is similar to the guidewire 310 illustrated in FIG. 8, except as described below. Thus, the guidewire 410 includes an elongate member 412 having proximal 414 and distal 420 ends. A main body 416 extends between the proximal 414 and distal 420 ends and has a diameter 418. A distal tip portion 421 is disposed adjacent a taper portion 424. The distal tip portion 421 comprises an elongate tip of a second, substantially uniform diameter and comprises the distal end 420 of the elongate member 412. The taper portion 424 is disposed between the main body 416 and the distal end 420 and defines a taper 426 between the first diameter 418 and the diameter of the distal tip portion 421. A coating 428 is disposed on the distal end 420 and comprises a flexible tip 430 that has a length that axially extends beyond the distal end 420 of the elongate member 412.

In this embodiment, the flexible tip 430 includes a curvilinear portion 450. The curvilinear portion 450 can define any suitable curve, and the actual curve chosen will depend on at least the application to which the guidewire 410 will be applied. As illustrated in FIG. 9, the curvilinear portion 450 can comprise a J-shape. This configuration may be suitable for use of the guidewire 410 in applications that may require a protective atraumatic tip, or a distal end suitable for selection of a vessel at a vessel junction, i.e., a selection tip.

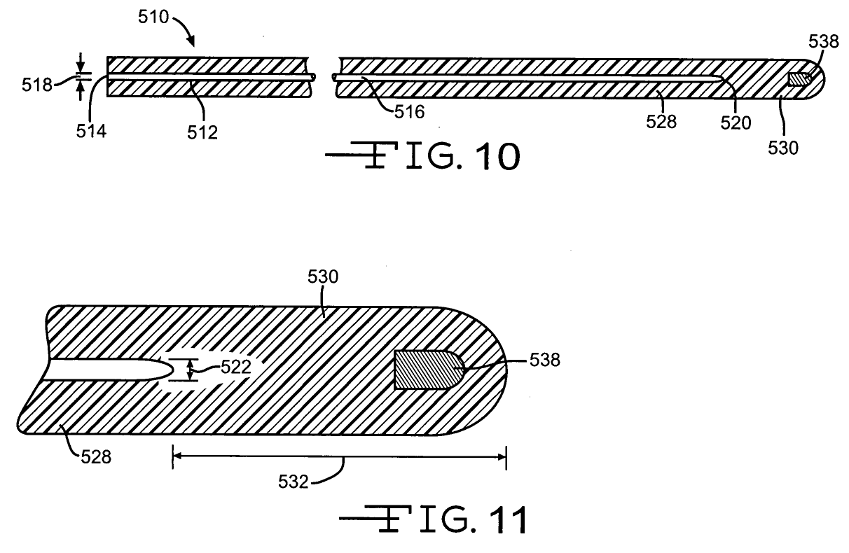

FIGS. 10 and 11 illustrate a guidewire 510 according to another embodiment of the invention. In this embodiment, the guidewire 510 comprises an elongate member 512 having proximal 514 and distal 520 ends. A main body 516 extends between the proximal 514 and distal 520 ends and has a diameter 518. The distal end 520 has a diameter 522 that may be the same as, larger than, or smaller than the diameter 518 of the main body 516. A coating 528 is disposed on the distal end 520 and at least a portion of the main body 516. The coating 528 comprises a flexible tip 530. The flexible tip 530 has a length 532 that axially extends beyond the distal end 520 of the elongate member 512. The ratio of the length 532 to the diameter 518 of the distal end 520 can be greater than 3:1. Also, the ratio of the length 532 to the diameter 518 can be between 10:1 and 500:1, between 10:1 and 300:1, and between 12:1 and 250:1. The length 532 should be greater than 3 mm for most guidewires, and can be between 3 and 100 mm, between 4 and 80 mm, between 5 and 20 mm, and between 8 and 12 mm. A 10 mm length is a particularly well suited length for a flexible tip on a guidewire for use in some vascular applications.

The coating 528 can have a radial thickness along a portion or the entire length of the main body 516 that is greater than the diameter 518 of the main body 516. This may provide enhanced rigidity to the guidewire 510.

A radiopaque marker 538 is disposed in the flexible tip 530 and spaced from the distal end 520 of the elongate member 512. A portion of the coating 528 can be disposed between the radiopaque marker 538 and the distal end 520 of the elongate member 512. The radiopaque marker 538 can be any suitable radiopaque marker, as described above.

The foregoing detailed description provides exemplary embodiments of the invention and includes the best mode for practicing the invention. These embodiments are intended only to serve as examples of the invention, and not to limit the scope of the invention in any manner.

1. A guidewire, comprising:
an elongate member comprising a proximal end, a main body having a first diameter, a distal end having a second diameter, and a taper portion disposed between the main body and distal end and defining a taper between the first and second diameters; and
a coating disposed on the distal end and comprising a flexible tip having a length axially extending beyond the distal end, the ratio of the length to the first diameter being greater than 3:1.

2. The guidewire of claim 1, further comprising a radiopaque marker disposed in the flexible tip.

3. The guidewire of claim 2, wherein a portion of the coating is disposed between the distal end of the elongate member and the radiopaque marker.

4. The guidewire of claim 1, wherein the flexible tip comprises radiopaque material.

5. The guidewire of claim 1, wherein the coating comprises radiopaque material.

6. The guidewire of claim 1, wherein the coating is disposed on at least a portion of the taper portion.

7. The guidewire of claim 6, wherein the coating is disposed on at least a portion of the main body.

8. The guidewire of claim 1, wherein the elongate member comprises one of stainless steel and nitinol.

9. The guidewire of claim 1, further comprising a lubricious coating disposed on at least a portion of the main body.

10. The guidewire of claim 1, wherein the ratio of the length of the flexible tip to the first diameter is between 10:1 and 500:1.

11. The guidewire of claim 1, wherein the ratio of the length of the flexible tip to the first diameter is between 10:1 and 300:1.

12. The guidewire of claim 1, wherein the ratio of the length of the flexible tip to the first diameter is between 12:1 and 250:1.

13. The guidewire of claim 1, wherein the length of the flexible tip is greater than 3 mm.

14. A guidewire, comprising:
an elongate member comprising a proximal end, a main body having a first diameter, a distal tip having a first length and a second diameter, and a taper portion disposed between the main body and distal tip and defining a taper between the first and second diameters;
a coating disposed on the distal tip and comprising a flexible tip having a second length axially extending beyond the distal tip, the second length being equal to or greater than the first length.

15. The guidewire of claim 14, further comprising a radiopaque marker disposed in the flexible tip.

16. The guidewire of claim 15, wherein a portion of the coating is disposed between the distal end and the radiopaque marker.

17. The guidewire of claim 14, wherein the flexible tip comprises radiopaque material.

18. The guidewire of claim 14, wherein the coating comprises radiopaque material.

19. The guidewire of claim 14, wherein the coating is disposed on at least a portion of the taper portion.

20. The guidewire of claim 19, wherein the coating is disposed on at least a portion of the main body.

21. A guidewire, comprising:
an elongate member comprising a proximal end, a main body having a first diameter, a distal tip having a second diameter, and a taper portion disposed between the main body and distal tip and defining a taper between the first and second diameters;
a polymer coating disposed on the distal tip and at least a portion of the taper portion, the polymer coating comprising a flexible tip having a length axially extending beyond the distal tip, the ratio of the length to the first diameter being greater than 3:1;
a radiopaque marker disposed in the flexible tip and spaced from the distal tip of the elongate member; and
a lubricious coating disposed on at least a portion of the main body.

22. The guidewire of claim 21, wherein the ratio of the length of the flexible tip to the first diameter is between 10:1 and 500:1

23. The guidewire of claim 21, wherein the ratio of the length of the flexible tip to the first diameter is between 10:1 and 300:1.

24. The guidewire of claim 21, wherein the ratio of the length of the flexible tip to the first diameter is between 12:1 and 250:1.

25. The guidewire of claim 21, wherein the length of the flexible tip is greater than 3 mm.

26. A guidewire, comprising:
an elongate member comprising proximal and distal ends; and
a coating disposed on the distal end of the elongate member, the coating comprising a flexible tip extending beyond the distal end and a solid member spaced from the distal end.

27. The guidewire according to claim 26, wherein the solid member comprises a radiopaque marker.

28. The guidewire according to claim 26, wherein a portion of the coating is disposed between the distal end of the elongate member and the solid member.
